(19) United States
(12) Patent Application Publication
(10) Pub. No.: US 2026/0315831 A1
(43) Pub. Date: Oct. 15, 2026

(54) ULTRASOUND OUTPUT DEVICE AND FOCUS CONTROL METHOD USING THE SAME

(71) Applicant: ALPINION MEDICAL SYSTEMS CO., LTD., Seoul (KR)

(72) Inventors: Jeongmin Heo, Seoul (KR); Susung Lee, Seoul (KR)

(21) Appl. No.: 19/484,349

(22) PCT Filed: May 28, 2024
(86) PCT No.: PCT/KR2024/007262

§ 371 Date: Nov. 13, 2025

(30) Foreign Application Priority Data
May 30, 2023 (KR) 10-2023-0068957

(51) Int. Cl. A61N 7/00

(52) U.S. Cl. CPC A61N 7/00; A61N 2007/0056

(57) ABSTRACT

Provided is to an ultrasound output device capable of controlling a focus, and the ultrasound output device capable of controlling a focus may include an ultrasound output unit including a plurality of output channels, and a control unit for controlling a focus generated by an ultrasound signal by controlling a delay time of the ultrasound signal output through the plurality of output channels and whether to activate the plurality of output channels.

### TECHNICAL FIELD

The present disclosure relates to an ultrasound output device and a focus control method using the same.

### BACKGROUND ART

Ultrasound treatment may be broadly divided into a method that uses high-intensity ultrasound of 1000 W/cm² or more to selectively heat tissue and treat tumors, and a method that uses low-intensity ultrasound of 10 to 50 W/cm² to heat subcutaneous tissue and treat musculoskeletal problems such as skin lifting, fractures, and cartilage cell regeneration. In addition, the ultrasound treatment may use focused ultrasound that focuses ultrasound signals on at least one focus of the target area.

The focus size of the focused ultrasound may be greatly influenced by the structure of a transducer or the frequency characteristics of the ultrasound signal, and may be determined by them. In other words, in order to control the focus size of the focused ultrasound, ultrasound of different frequencies should be used, and therefore, different transducers are required.

The technology underlying the present application is disclosed in Korean Patent No. 10-2005497.

### DISCLOSURE

### Technical Problem

The present disclosure is intended to solve the problems of the prior art described above, and an object thereof is to provide an ultrasound output device and a focus control method using the same, which can control the focus size of focused ultrasound and generate a plurality of foci by using only an ultrasound signal having one frequency component by controlling the delay time of ultrasound signals output through a plurality of output channels and whether or not the plurality of output channels are activated.

However, the technical tasks to be achieved by the embodiments of the present disclosure are not limited to the technical tasks described above, and other technical tasks may exist.

### Technical Solution

As a technical means for achieving the above-described technical task, an ultrasound output device capable of controlling a focus according to one embodiment of the present disclosure may include: an ultrasound output unit including a plurality of output channels; and a control unit for controlling a focus generated by an ultrasound signal by controlling a delay time of the ultrasound signal output through the plurality of output channels and whether to activate the plurality of output channels.

According to one embodiment of the present disclosure, the control unit may include a grouping unit that groups the plurality of output channels to generate a plurality of channel groups.

According to one embodiment of the present disclosure, the control unit may include a multi-focus control unit that controls the delay time of a plurality of ultrasound signals output from each of the plurality of channel groups to generate a plurality of foci.

According to one embodiment of the present disclosure, the control unit may include a focus size control unit that controls the delay time of the plurality of ultrasound signals output from each of the plurality of channel groups to generate at least one focus and controls the focus size of the at least one focus by controlling whether to activate each of the plurality of channel groups.

According to one embodiment of the present disclosure, the focus size control unit may include a prediction unit that predicts a prediction focus size when at least one channel group is activated based on angle information for the at least one channel group among the plurality of channel groups, and a determination unit that determines whether to activate each of the plurality of channel groups based on the prediction focus size.

According to one embodiment of the present disclosure, when any one channel group o the plurality of channel groups is activated, the angle information may be a focus angle for the any one channel group, and when at least two channel groups of the plurality of channel groups are activated, the angle information may be a sum of at least two focus angles for each of the at least two channel groups.

According to one embodiment of the present disclosure, the determination unit may set a plurality of focus size levels based on the angle information, and determine whether to activate each of the plurality of channel groups based on any one focus size level of the plurality of focus size levels corresponding to a target focus size.

According to one embodiment of the present disclosure, the focus size control unit may further include a pressure maintenance unit that maintains a pressure intensity of the ultrasound signal within a preset range depending on whether to activate each of the plurality of channel groups.

As a technical means for achieving the above-mentioned technical task, an ultrasound focus control method may include controlling a focus generated by an ultrasound signal by controlling a delay time of the ultrasound signal output through a plurality of output channels and whether to activate the plurality of output channels.

According to one embodiment of the present disclosure, the controlling of the focus may include grouping the plurality of output channels to generate a plurality of channel groups, controlling the delay time of the ultrasound signal output from each of the plurality of channel groups to generate a plurality of foci, and controlling the delay time of the ultrasound signal output from each of the plurality of channel groups to generate at least one focus, and controlling the focus size of the at least one focus by controlling whether to activate each of the plurality of channel groups.

According to one embodiment of the present disclosure, the controlling of the focus size may include predicting a prediction focus size when at least one channel group is activated based on angle information for the at least one channel group among the plurality of channel groups, determining whether to activate each of the plurality of channel groups based on the prediction focus size, and maintaining a pressure intensity of the ultrasound signal within a preset range depending on whether to activate each of the plurality of channel groups.

The means for solving the above-described problems are merely exemplary and should not be construed as limiting the present disclosure. In addition to the above-described exemplary embodiments, additional embodiments may exist in the drawings and detailed description of the invention.

### Advantageous Effects

According to the above-described means for solving the problems of the present disclosure, the focus size can be controlled based on the focus angle of the ultrasound signal by controlling whether to activate the ultrasound signal output from each of the plurality of output channels.

According to the above-described means for solving the problems of the present disclosure, the plurality of foci can be generated by controlling the delay time of the ultrasound signals output from each of the plurality of output channels.

According to the above-described means for solving the problems of the present disclosure, by controlling the delay time of the ultrasound signals output through the plurality of output channels and whether to activate the plurality of output channels, it is possible to control the focus size of the focused ultrasound and generate the plurality of foci by using only an ultrasound signal having a single frequency component without the use of additional transducers.

According to the above-described means for solving the problems of the present disclosure, it is possible to control the focus size of the focused ultrasound and generate the plurality of foci by using only the ultrasound signal having a single frequency component. This allows the position, number, and size of foci to be adjusted for various purposes, thereby improving treatment efficiency and reducing treatment time.

However, the effects achieved by the present disclosure are not limited to the aforementioned effects, and other effects may also exist.

### DESCRIPTION OF DRAWINGS

FIG. 1 is a schematic block diagram of an ultrasound output device according to one embodiment of the present disclosure.

FIG. 2 is a diagram illustrating beam steering for focusing an ultrasound signal according to one embodiment of the present disclosure.

FIG. 3 is a diagram illustrating an example of grouping a plurality of output channels according to one embodiment of the present disclosure.

FIGS. 4 and 5 are diagrams illustrating delay times of ultrasound signals and experimental examples thereof for explaining the generation of a plurality of foci according to one embodiment of the present disclosure.

FIGS. 6 and 7 are diagrams illustrating an experimental example of focus size control according to activated channel groups of the plurality of output channels according to one embodiment of the present disclosure.

FIG. 8 is a diagram illustrating the focus angle of ultrasound signals according to the activated channel groups of the plurality of output channels according to one embodiment of the present disclosure.

FIG. 9 is a table illustrating an experimental example of focus angle and focus size of the ultrasound signals according to the activated channel groups of the plurality of output channels according to one embodiment of the present disclosure.

FIG. 10 is a table illustrating an example of setting focus size levels according to the activated channel groups of the plurality of output channels according to one embodiment of the present disclosure.

FIG. 11 is a diagram illustrating an experimental example of pressure intensity according to the activated channel groups of the plurality of output channels according to one embodiment of the present disclosure.

FIG. 12 is an operational flowchart of a focus control method of an ultrasound output device according to one embodiment of the present disclosure.

FIG. 13 is an operational flowchart illustrating a method for controlling the focus size of an ultrasound output device according to one embodiment of the present disclosure.

### BEST MODE

Below, with reference to the attached drawings, embodiments of the present disclosure are described in detail so that those skilled in the art can easily practice them. However, the present disclosure may be implemented in various different forms and is not limited to the embodiments described herein. Furthermore, for clarity in the drawings, portions irrelevant to the description have been omitted, and similar parts are designated with similar reference numerals throughout the specification.

Throughout this specification, when a part is referred to as being “connected” to another part, this includes not only “directly connected” but also “electrically connected” or “indirectly connected” with another element intervening.

Throughout this specification, when a member is referred to as being “on”, “above”, “at an upper end of”, “below”, “under”, or “at a lower end of” another member, this includes not only cases where the member is in contact with the other member, but also cases where another member exists between the two members.

Throughout this specification, when a part is said to “include” a component, this does not mean that other components are excluded, but rather that other components may be included, unless otherwise specifically stated.

The present disclosure relates to an ultrasound output device and a focus control method using the same.

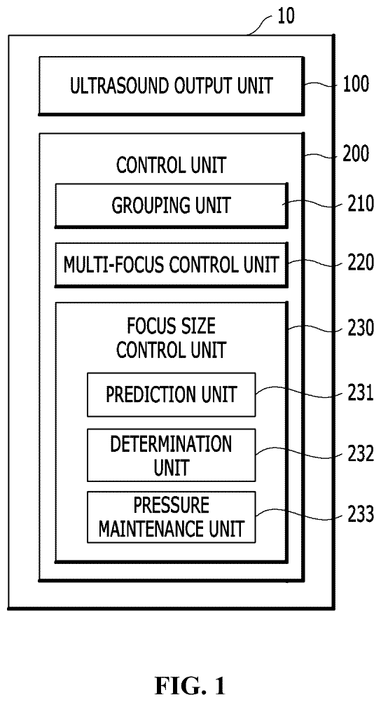

FIG. 1 is a schematic block diagram of an ultrasound output device according to one embodiment of the disclosure.

Referring to FIG. 1, an ultrasound output device 10 may include an ultrasound output unit 100 and a control unit 200. Furthermore, the control unit 200 may include a grouping unit 210, a multi-focus control unit 220, and a focus size control unit 230, and the focus size control unit 230 may include a prediction unit 231, a determination unit 232, and a pressure maintenance unit 233.

According to one embodiment of the present disclosure, the ultrasound output unit 100 may refer to an ultrasound probe. Specifically, the ultrasound output unit 100 may include a transducer T including a plurality of output channels, and each of the plurality of output channels may include at least one piezoelectric element.

That is, the transducer T may be a phased array transducer including a plurality of piezoelectric elements arranged in an array, and may be configured to convert a time-delayed electric signal applied to each of the plurality of piezoelectric elements into a vibration signal to perform beam forming.

According to one embodiment of the present disclosure, the control unit 200 may refer to a central processing unit that generally controls the operation of the ultrasound output device 10, including the ultrasound probe or the transducer within the ultrasound probe.

In other words, the control unit 200 may be implemented using, for example, at least one of Application Specific Integrated Circuits (ASICs), Digital Signal Processors (DSPs), Digital Signal Processing Devices (DSPDs), Programmable Logic Devices (PLDs), Field-Programmable Gate Arrays (FPGAs), processors, controllers, microcontrollers, and microprocessors, and may be implemented as a firmware/software module executable on the aforementioned hardware platform. In this case, the firmware/software module may be implemented by one or more software applications written in an appropriate program language.

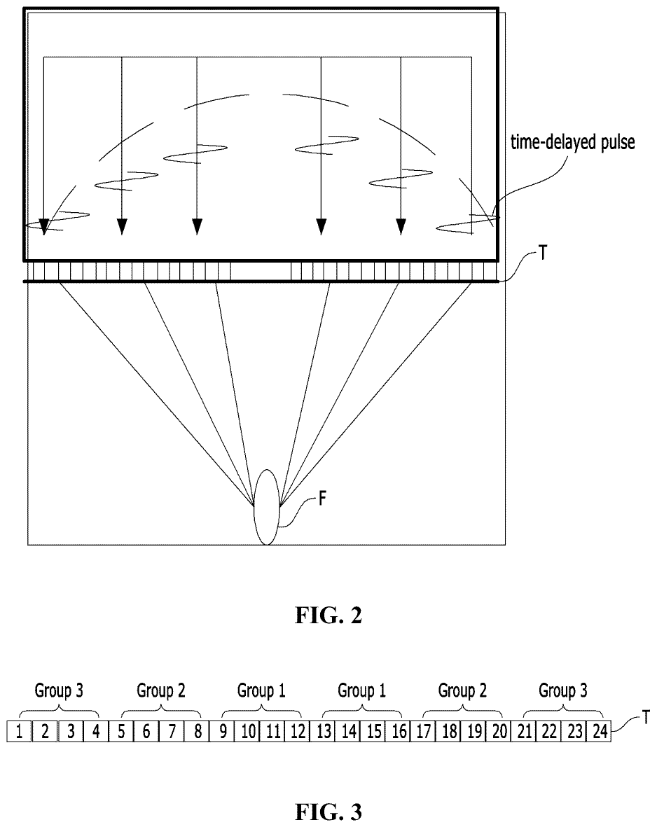

FIG. 2 is a diagram illustrating beam steering for focusing the ultrasound signal according to one embodiment of the present disclosure.

Referring to FIG. 2, the control unit 200 may focus the ultrasound beam on a target object at a focus F by adjusting the delay time of a pulse for each of the plurality of piezoelectric elements. In other words, the control unit 200 may control the delay time of ultrasound signals output through the plurality of output channels of the ultrasound output unit 100 to generate the focus F.

In addition, the control unit 200 may control the delay time of the ultrasound signal output through the plurality of output channels, and may also control whether to activate each of the plurality of output channels. Furthermore, the control unit 200 may control the plurality of output channels and the ultrasound signals output through the plurality of output channels to generate the focus, and control the position, number, and size of the generated focus.

Below, the configuration of the control unit 200 for controlling the focus according to one embodiment of the present disclosure will be described in detail.

FIG. 3 is a diagram illustrating an example of grouping the plurality of output channels according to one embodiment of the present disclosure.

Referring to FIG. 3, the grouping unit 210 may group the plurality of output channels of the ultrasound output unit 100 to generate a plurality of channel groups. Specifically, when the transducer T is expressed on a plane, the grouping unit 210 may group a predetermined number of output channels, starting from the output channel closest to the center of the transducer T, to generate the channel groups. Furthermore, the grouping unit may group the same number of output channels on both sides of the plane based on the center of the transducer T to generate pairs of channel groups.

In this case, the plurality of output channels may be arranged in a substantially circular, oval, or rectangular shape. FIG. 3 illustrates any one aspect of the transducer T including the plurality of output channels arranged in various shapes. The both sides may refer to areas according to the arrangement shape of the plurality of output channels, such as a circle, an oval, or a square, based on the center of the transducer T.

Referring to FIG. 3, the grouping unit 210 may group four output channels from the center of the transducer T into one group, thereby generating a Group 1 G1, a Group 2 G2, and a Group 3 G3 that are symmetrical about the center. However, the present disclosure is not limited thereto, and the number of groups and the number of output channels included in each group may be determined differently depending on the purpose and environment of use of the transducer T, user settings, or the like. In the following, for the convenience of explanation, the description will proceed assuming that the plurality of channel groups according to one embodiment of the present disclosure includes the Group 1 G1, the Group 2 G2, and the Group 3 G3 exemplified in FIG. 3, but is not limited thereto.

In this regard, the control unit 200 may control the ultrasound signals output from each of the plurality of channel groups. In other words, the control unit 200 may control the output signals of each of the plurality of output channels, and may also control the output signals for each of the plurality of channel groups. That is, the control unit 200 may control the plurality of output channels included in one channel group to be used as a single output channel by linking them with each other.

Furthermore, the control unit 200 may control the delay time of the plurality of ultrasound signals output through at least one channel group of the plurality of channel groups to generate at least one focus where the ultrasound signals are focused. In this case, the control unit 200 may selectively use only any one of the plurality of channel groups, or may selectively use at least two of the plurality of channel groups.

According to one embodiment of the present disclosure, the multi-focus control unit 220 may generate the plurality of foci by controlling the delay time of the plurality of ultrasound signals output from each of the plurality of channel groups.

Specifically, the multi-focus control unit 220 may generate the plurality of foci by controlling the delay time of the ultrasound signal output through any one of the plurality of channel groups to focus the corresponding ultrasound signal at any one position to generate the focus, and by controlling the delay time of the ultrasound signal output through another of the plurality of channel groups to focus the corresponding ultrasound signal at another position to generate the focus.

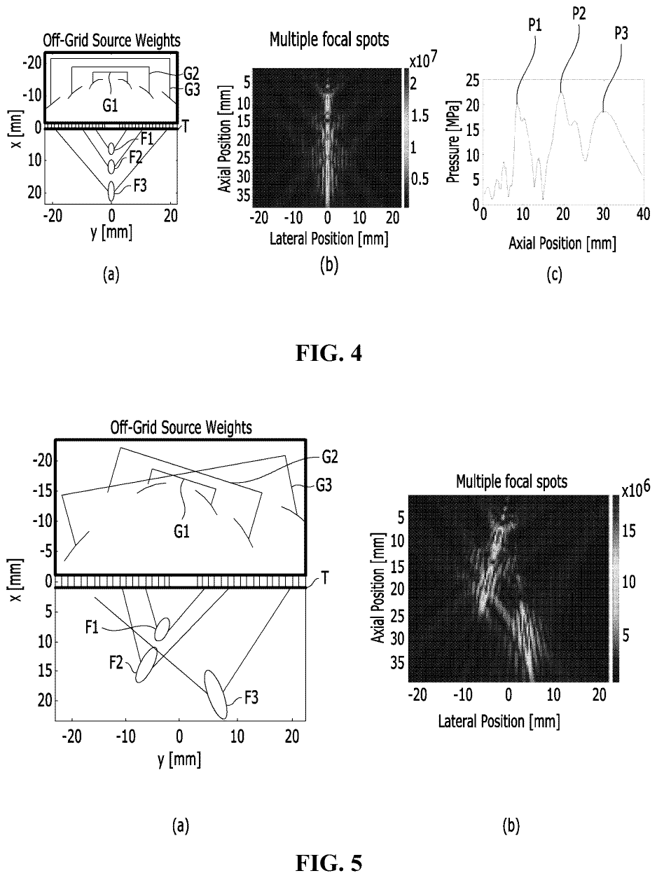

FIGS. 4 and 5 are diagrams illustrating the delay time of an ultrasound signal and an experimental example thereof for explaining the generation of the plurality of foci according to one embodiment of the present disclosure.

Specifically, FIG. 4 is a diagram illustrating the generation of the plurality of foci F1, F2, and F3 corresponding to the Group 1 G1, the Group 2 G2, and the Group 3 G3 on the same axis (axial axis) by controlling the delay time of the ultrasound signal corresponding to each of the Group 1 G1, the Group 2 G2, and the Group 3 G3. In FIG. 4, the coordinates of the first focus F1 for the Group 1 G1 may be [0 mm, 10 mm], the coordinates of the second focus F2 for the Group 2 G2 may be [0 mm, 20 mm], and the coordinates of the third focus F3 for the Group 3 G3 may be [0 mm, 30 mm].

In addition, FIG. 5 is a diagram illustrating the generation of the plurality of foci F1, F2, and F3 corresponding to the Group 1 G1, the Group 2 G2, and the Group 3 G3 by controlling the delay time of the ultrasound signals corresponding to each of the Group 1 G1, the Group 2 G2, and the Group 3 G3 so as to focus each ultrasound signal at different positions, for example, on different axes. In FIG. 5, the coordinates of the first focus F1 for the Group 1 G1 may be [−3 mm, 10 mm], the coordinates of the second focus F2 for the Group 2 G2 may be [−5 mm, 20 mm], and the coordinates of the third focus F3 for the Group 3 G3 may be [5 mm, 30 mm].

(a) of FIG. 4 and (a) of FIG. 5 illustrate the delay time of the ultrasound signal for each channel group and the position of each focus generated by the ultrasound signal, and (b) of FIG. 4 and (b) of FIG. 5 illustrate experimental examples for (a) of FIG. 4 and (a) of FIG. 5, respectively. In addition, (c) of FIG. 4 is a graph illustrating the position and pressure intensity on the axial axis of each focus.

Referring to FIGS. 4 and 5, the multi-focus control unit 220 may adjust the position and depth of focus corresponding to each channel ultrasound signal by controlling the delay time differently for each channel ultrasound signal of each of the plurality of channel groups.

In this case, referring to FIGS. 4 and 5, it can be confirmed that the axial focus size increases as the focal length (the distance from the transducer to the focus) increases. This is due to the acoustic focusing intensity. When the focal length is long, the focusing intensity is low, so the focus size is formed relatively large, and conversely, when the focal length is short, the focusing intensity is high, so a relatively small focus size is formed. In addition, since the focus size increases as the focal length increases, the pressure intensity relative to the focal volume decreases. However, there is a problem that the focus size at the same focal length cannot be adjusted only with the characteristic that the focus size changes according to the focal length.

In this regard, the focus size of focused ultrasound may generally be related to the frequency characteristics, focus angle, and characteristics of the ultrasound propagation medium. The control unit 200 may control the focus size of the focus based on the change in focus size according to the focus angle of the ultrasound signal among them.

According to one embodiment of the present disclosure, the focus size control unit 230 may generate at least one focus by controlling the delay time of the plurality of ultrasound signals output from each of the plurality of channel groups, and may control the focus size of at least one focus by controlling whether to activate each of the plurality of channel groups.

Here, the delay time control of the plurality of channel groups performed by the focus size control unit 230 may be the same process as that performed by the multi-focus control unit 220, but may be described separately for convenience of explanation.

Specifically, the focus size control unit 230 may generate at least one focus using at least one channel group. In other words, the focus size control unit 230 may focus the first ultrasound signal output through the first group on a first position to generate a first focus, and the second ultrasound signal output through the second group on a second position to generate a second focus. However, the present disclosure is not limited thereto, and may focus both the first ultrasound signal and the second ultrasound signal on the first position to generate a single focus using two channel groups.

In this case, even when the focus is generated at the same position, the focus size of the focus generated by a single ultrasound signal and the focus size of the focus generated by the plurality of ultrasound signals may be different from each other.

In other words, the focus size control unit 230 may control the focus size by selectively controlling whether to activate the plurality of channel groups that output ultrasound signals focused on the position, that is, each ultrasound signal, when generating a focus at any one position.

Below, the configuration of the focus size control unit 230 for controlling the focus size according to one embodiment of the present disclosure will be described in detail.

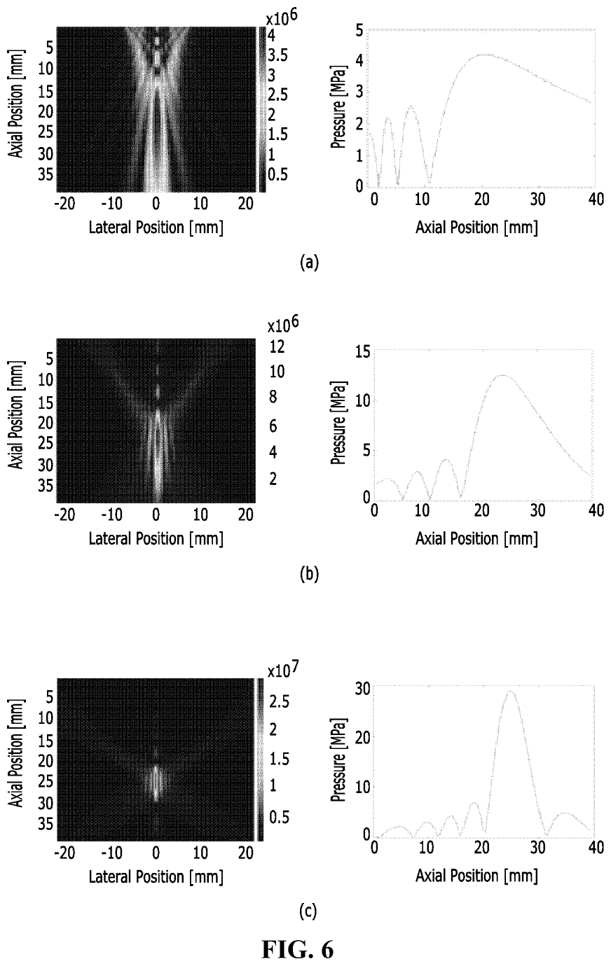

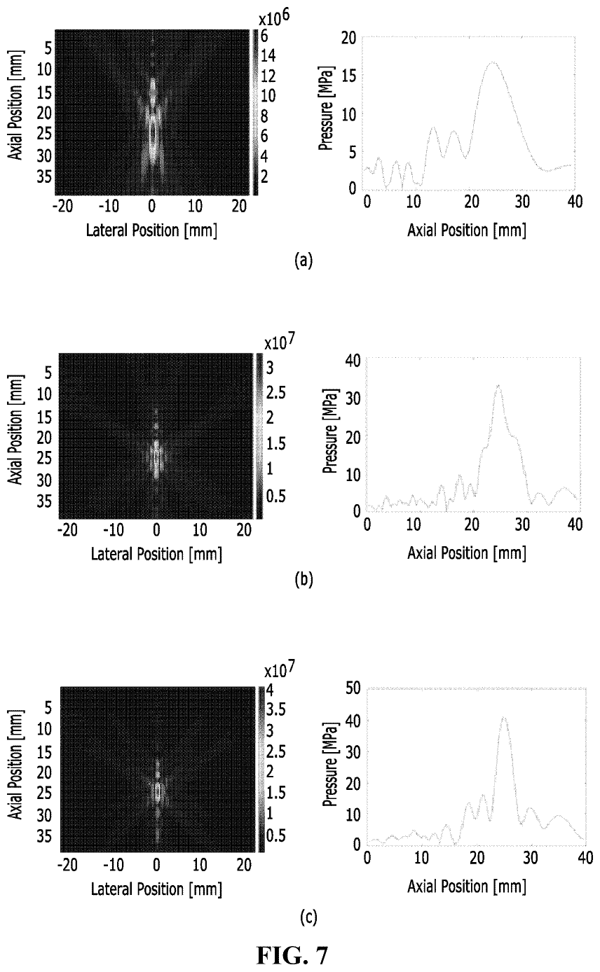

FIGS. 6 and 7 are diagrams illustrating experimental examples of focus size control according to activated channel groups of the plurality of output channels according to one embodiment of the present disclosure. In this case, the foci illustrated in (a) of FIG. 6 to (c) of FIG. 6 and (a) of FIG. 7 to (c) of FIG. 7 are all focused at the same focus depth and position, and the corresponding focus depth may be, for example, 25 mm.

Specifically, FIG. 6 illustrates the focus generated by focusing one ultrasound signal output through one channel group. (a) of FIG. 6 illustrates the focus size and pressure intensity when only the Group 1 G1 is activated, (b) of FIG. 6 illustrates the focus size and pressure intensity when only the Group 2 G2 is activated, and (c) of FIG. 6 illustrates the focus size and pressure intensity when only the Group 3 G3 is activated.

FIG. 7 illustrates a focus generated by focusing two ultrasound signals output through two channel groups at the same position. (a) of FIG. 7 illustrates the focus size and pressure intensity when the Group 1 G1 and the Group 2 G2 are activated, (b) of FIG. 7 illustrates the focus size and pressure intensity when the Group 1 G1 and the Group 3 G3 are activated, and (c) of FIG. 7 illustrates the focus size and pressure intensity when the Group 2 G2 and the Group 3 G3 are activated.

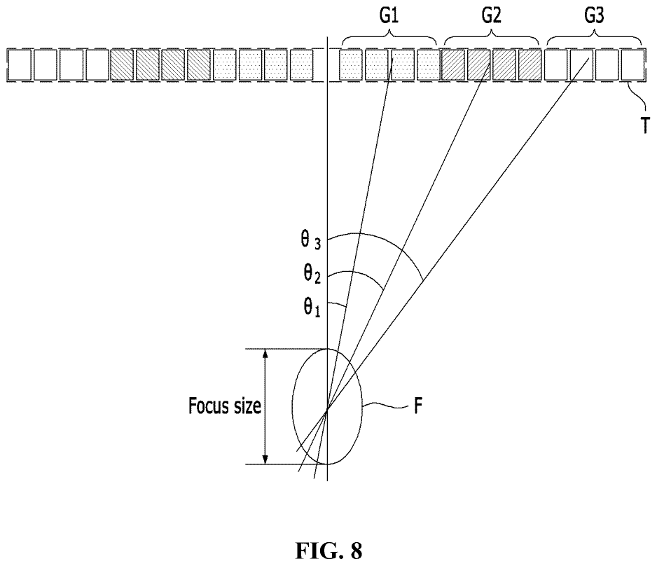

FIG. 8 is a diagram illustrating a focus angle of an ultrasound signal according to the activated channel group of the plurality of output channels according to one embodiment of the present disclosure. Referring to FIG. 8, the focus angle may mean an angle between a connecting line from the center point of each channel group to the focus and a vertical axis (axial axis) of the focus. However, the present disclosure is not limited thereto, and when the focus is located at a position off the vertical axis with respect to the center of the transducer T, the focus angle may be determined based on another vertical axis that passes through the focus and is parallel to the vertical axis with respect to the center of the transducer T.

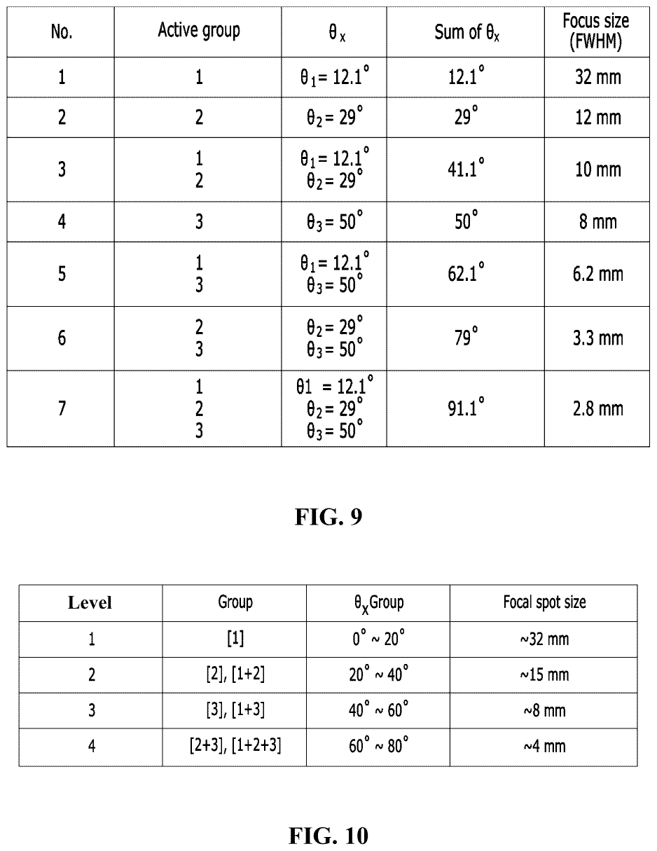

FIG. 9 is a table illustrating an experimental example of the focus angle and focus size of the ultrasound signal according to the activated channel group of the plurality of output channels according to one embodiment of the present disclosure. In other words, FIG. 9 may be a table that summarizes the experimental example of FIGS. 6 and 7 in relation to the focus angle according to the activated channel group.

Referring to FIGS. 6, 8, and 9, when only the Group 1 G1 is activated, it can be confirmed that the focus angle is 12.1° and the axial focus size is approximately 32 mm. In addition, when only the Group 2 G2 is activated, it can be confirmed that the focus angle is 29° and the focus size is approximately 12 mm, and when only the Group 3 G3 is activated, it can be confirmed that the focus angle is 50° and the focus size is approximately 8 mm.

Moreover, referring to FIGS. 7, 8, and 9, when the Group 1 G1 and the Group 2 G2 are activated, it can be confirmed that the focus angles are 12.1° and 29°, respectively, and the focus size is about 10 mm. Also, when the Group 1 G1 and the Group 3 G3 are activated, it can be confirmed that the focus angles are 12.1° and 50°, respectively, and the focus size is about 6.2 mm, and when the Group 2 G2 and the Group 3 G3 are activated, it can be confirmed that the focus angles are 29° and 50°, respectively, and the focus size is about 3.3 mm.

Also, referring to FIG. 9, when the Group 1 G1, Group 2 G2, and Group 3 G3 are all activated, it can be formed that the focus angles are 12.1°, 29°, and 50°, respectively, and the focus size is approximately 2.8 mm.

According to FIGS. 6 to 9, the focus of the focused ultrasound may be larger when only one channel group is activated compared to when the plurality of channel groups is activated, and may be larger when the focus angle of the ultrasound signal is small compared to when the focus angle is large.

That is, referring to FIGS. 6 to 9, the focus size may vary depending on the focus angle of the ultrasound signal output through the activated channel group, allowing the focus angle and focus size to be determined based on the activated channel group.

As described above, the focus size control unit 230 according to one embodiment of the present disclosure may predict the focus angle and focus size according to the activated channel group, and determine the channel groups to be activated to generate the focus with the target focus size based on the predicted focus size.

However, when the plurality of channel groups is activated rather than a single channel group, a plurality of focus angles exists, which may cause confusion in predicting the focus size according to the activated channel group. Therefore, to clarify the relationship between whether to activate the plurality of channel groups, the focus angle for each channel group, and the focus size of the generated focus, the focus size control unit 230 may consider the sum of the focus angles for each of the plurality of channel groups when predicting the focus size when the plurality of channel groups is activated.

In other words, the prediction unit 231 of the focus size control unit 230 may predict the prediction focus size when at least one channel group is activated based on angle information for at least one channel group among the plurality of channel groups. In this case, when one channel group of the plurality of channel groups is activated, the angle information may be the focus angle for the one channel group, and when at least two channel groups of the plurality of channel groups are activated, the angle information may be the sum of at least two focus angles for each of the at least two channel groups.

In addition, the determination unit 232 of the focus size control unit 230 may determine and control whether to activate each of the plurality of channel groups based on the prediction focus size predicted by the prediction unit 231.

Specifically, when predicting the focus size in a case where a first ultrasound signal output through the Group 1 G1 is delayed by a first time and the second ultrasound signal output through the Group 2 G2 is delayed by a second time to generate a focus at a predetermined position, the prediction unit 231 may determine a first focus angle of the first ultrasound signal for the predetermined position and a second focus angle of the second ultrasound signal for the predetermined position according to the first time and the second time, and predict the focus size according to the first focus angle and the second focus angle. In addition, the prediction unit 231 may also predict the focus size in a case where the Group 1 G1 and the Group 3 G3 are activated in the same manner.

In this case, when the focus size for activating the Group 1 G1 and the Group 2 G2 is referred to as the first focus size, and the focus size for activating the Group 1 G1 and the Group 3 G3 is referred to as the second focus size, the determination unit 232 may compare the first focus size and the second focus size with the target focus size to determine the focus size more suitable for a predetermined position, and select and activate a channel group that can generate a focus with the determined focus size.

That is, the prediction unit 231 may predict the focus sizes according to a channel group combination including at least one channel group among the plurality of channel groups, and the determination unit 232 may activate at least one channel group that can generate a focus size most similar to the target focus size for a predetermined position among the predicted focus sizes.

According to one embodiment of the present disclosure, the control unit 200 may perform focus size control of focused ultrasound and generation of the plurality of foci using only an ultrasound signal having a single frequency component without utilizing an additional transducer by controlling the delay time of ultrasound signals output through the plurality of output channels and whether to activate the plurality of output channels.

In addition, the control unit 200 may control the focus size of the focused ultrasound and generate the plurality of foci using only an ultrasound signal having a single frequency component, thereby adjusting the position, number, and size of the foci for various purposes, thereby improving treatment efficiency and reducing treatment time.

In one embodiment of the present disclosure, the determination unit 232 may set a plurality of focus size levels based on the angle information, and determine whether to activate each of the plurality of channel groups based on any one focus size level corresponding to the target focus size among the plurality of focus size levels.

FIG. 10 is a table illustrating an example of setting the focus size level according to the activated channel group of the plurality of output channels according to one embodiment of the present disclosure.

The focus size level may be classified based on a predetermined range of the angle information and focus size for the focus that may be generated according to the combination of activation groups, and as an example with reference to FIG. 10, the focus size level may include 4 levels (steps) classified by 20° each of angle information from 0° to 80°, and may be classified and determined based on the purpose and environment of use of the focused ultrasound, user settings, or the like.

Specifically, the determination unit 322 may generate and store the level table based on the prediction focus sizes according to whether to activate the plurality of channel groups as predicted by the prediction unit 321, and, when receiving the user input corresponding to any one of the plurality of focus size levels included in the level table, determine whether to activate each of the plurality of channel groups based on the stored level table.

As described above, the focus size control unit 230 generates and stores the level table, thereby reducing the computational process for predicting the focus sizes based on the activation group combinations and determining whether to activate the plurality of channel groups to generate the focus corresponding to the target focus size. Accordingly, since the computational process for the predetermined position for generating the focus, the focus angle for each channel group, and the focus size based on angle information according to the focus angle can be reduced, the efficiency in terms of time and energy for focus size control may be improved.

According to one embodiment of the present disclosure, the pressure maintenance unit 233 may maintain the pressure intensity of the ultrasound signal within a preset range depending on whether each of the plurality of channel groups is activated.

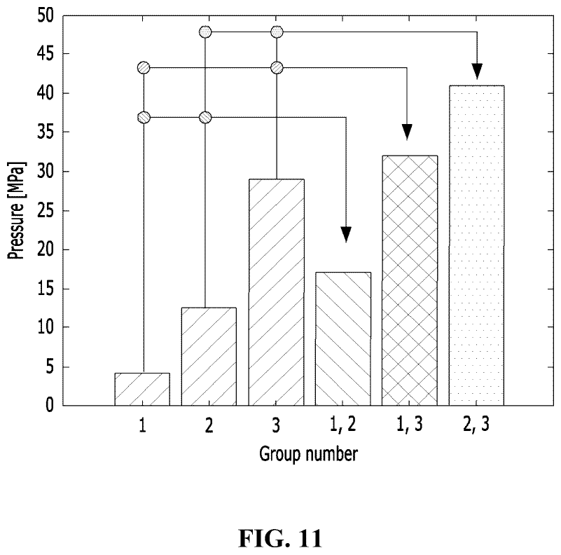

FIG. 11 is a diagram illustrating an experimental example of the pressure intensity according to the activated channel group of the plurality of output channels according to one embodiment of the present disclosure.

Referring to FIG. 11, it can be confirmed that when the pressure intensity of the ultrasound signal when the Group 1 G1 is activated is 4.1 MPa, the pressure intensity of the ultrasound signal when the Group 2 G2 is activated is 12.5 MPa, and the pressure intensity of the ultrasound signal when the Group 3 G3 is activated is 29 MPa, the pressure intensity of the ultrasound signal when the Group 1 G1 and the Group 2 G2 are activated is 17 MPa, the pressure intensity of the ultrasound signal when the Group 1 G1 and the Group 3 G3 are activated is 32.4 MPa, and the pressure intensity of the ultrasound signal when the Group 2 G2 and the Group 3 G3 are activated is 41 MPa.

That is, referring to FIG. 11, when the plurality of channel groups is activated, the pressure intensity of the ultrasound signal output through the activated channel groups may be similar to the pressure intensity of the ultrasound signal for each of the activated channel groups. (4.1(G1)+12.5(G2)≈17(G1,G2), 4.1(G1)+29(G3)≈32.4(G1,G3), 12.5(G2)+29(G3)≈41(G2,G3)).

The pressure maintenance unit 233, considering the matters illustrated in FIG. 11, may be configured to predict the pressure intensity of the ultrasound signal focused on the focus when controlling the focus size, and compensate for the pressure intensity that varies according to the change in the focus size by adjusting the intensity of the electrical signal.

In addition, the pressure maintenance unit 233 may be configured to adjust the intensity of the electrical signal so that the total pressure intensity of the ultrasound signal is maintained within a preset range, and when the total pressure intensity of the ultrasound signal exceeds the preset range, the intensity of the electrical signal may be lowered, and when the total pressure intensity of the ultrasound signal is below the preset range, the intensity of the electrical signal may be increased.

In other words, the focus size control unit 230 obtains pressure information (information on the pressure intensity of the ultrasound signal output through each channel group) for each of the plurality of channel groups, determines whether to activate the plurality of channel groups based on the prediction focus size according to the combination of activation groups, and controls the focus size of the focus generated at a predetermined position. Furthermore, the focus size control unit 230 calculates the pressure intensity of the ultrasound signal output through the activated channel groups based on the pressure information for each of the plurality of channel groups, and increases or decreases the intensity of the electrical signal applied to the plurality of channel groups based on the calculation result, thereby controlling the pressure intensity so that the pressure intensity remains within a preset range.

Accordingly, the focus size control unit 230 may control only the focus size while maintaining the total pressure intensity of the ultrasound signal focused on the focus constant. Therefore, when controlling the focus size, the focus and the area surrounding the focus may be prevented from being damaged by excessive increases in the pressure intensity of the ultrasound signal, thereby enabling safe treatment, and preventing a decrease in treatment efficiency due to insufficient pressure intensity.

Below, based on the detailed description above, the operational flow of the present disclosure will be briefly reviewed.

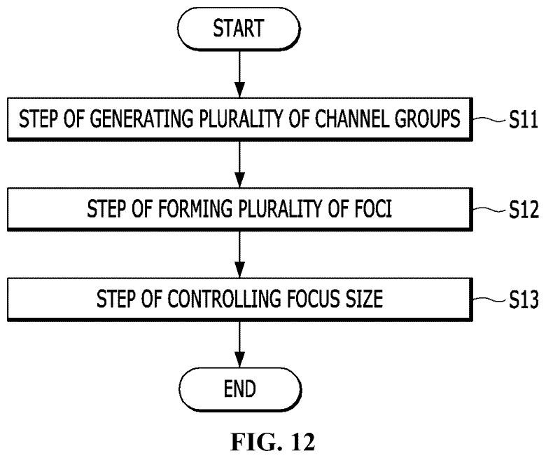

FIG. 12 is an operational flowchart of a focus control method of the ultrasound output device according to one embodiment of the present disclosure.

FIG. 12 is an operational flowchart of the focus control method according to one embodiment of the present disclosure.

The focus control method illustrated in FIG. 12 may be performed by the ultrasound output device 10 described above. Therefore, even if omitted below, the description of the ultrasound output device 10 may be equally applied to the description of the focus control method.

An ultrasound output device 10 according to one embodiment of the present disclosure may include the ultrasound output unit 100 including the plurality of output channels, and the control unit 200 that controls the delay time of the ultrasound signal output through the plurality of output channels and whether to activate the plurality of output channels, thereby controlling the focus generated by the ultrasound signals. In addition, the control unit 200 may include the grouping unit 210, the multi-focus control unit 220, and the focus size control unit 230.

Referring to FIG. 12, in Step S11, the grouping unit 210 may group the plurality of output channels to generate the plurality of channel groups.

Next, in Step S12, the multi-focus control unit 220 may control the delay times of the plurality of ultrasound signals output from each of the plurality of channel groups to generate the plurality of foci for each of the plurality of channel groups.

Next, in Step S13, the focus size control unit 230 may control the delay times of the ultrasound signals output from each of the plurality of channel groups to generate at least one focus, and control the focus size of the at least one focus by controlling whether to activate each of the plurality of channel groups.

More specifically, the focus size control unit 230 may include the prediction unit 231, the determination unit 232, and the pressure maintenance unit 233.

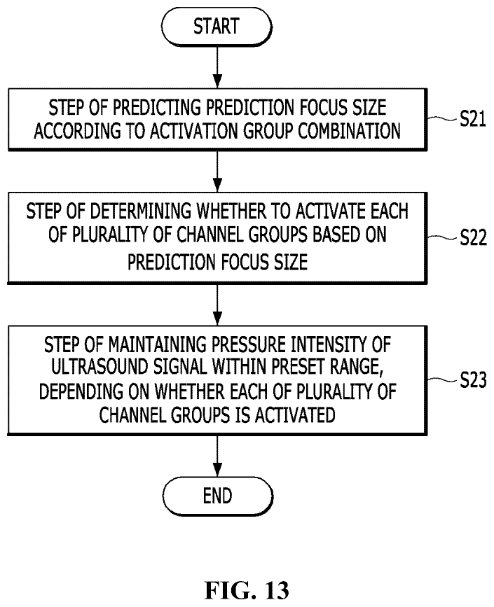

FIG. 13 is an operational flowchart illustrating the focus size control method of the ultrasound output device according to one embodiment of the present disclosure.

The focus size control method illustrated in FIG. 13 may be performed by the ultrasound output device 10 described above. Therefore, even when omitted below, the description of the ultrasound output device 10 may be equally applied to the description of the focus size control method.

Referring to FIG. 13, in Step S21, the prediction unit 231 may predict the prediction focus size when at least one channel group among the plurality of channel groups is activated based on the angle information for the at least one channel group among the plurality of channel groups. At this time, when one channel group of the plurality of channel groups is activated, the angle information may be the focus angle for the one channel group, and when at least two channel groups of the plurality of channel groups are activated, the angle information may be the sum of at least two focus angles for each of the at least two channel groups.

Next, in Step S22, the determination unit 232 may determine whether to activate each of the plurality of channel groups based on the prediction focus size. Specifically, in Step S22, the determination unit 232 may set the plurality of focus size levels based on angle information, and determine whether to activate each of the plurality of channel groups based on one focus size level corresponding to the target focus size among the plurality of focus size levels.

Next, in Step S23, the pressure maintenance unit 233 may maintain the pressure intensity of the ultrasound signal within a preset range, depending on whether each of the plurality of channel groups is activated.

In the above descriptions, Steps S11 to S13 and Steps S21 to S23 may be further divided into additional steps or combined into fewer steps, depending on the implementation example of the present disclosure. Furthermore, some steps may be omitted as needed, or the order of the steps may be changed.

The focus control method and focus size control method according to one embodiment of the present disclosure may be implemented in the form of program instructions that can be executed by various computer means and recorded on a computer-readable medium. The computer-readable medium may include program instructions, data files, data structures, and the like, either singly or in combination. The program instructions recorded on the medium may be specifically designed and configured for the present disclosure, or may be known and usable by those skilled in the art of computer software. Examples of computer-readable recording media include magnetic media such as hard disks, floppy disks, and magnetic tapes, optical media such as CD-ROMs and DVDs, magneto-optical media such as floptical disks, and hardware devices specifically configured to store and execute program instructions, such as ROMs, RAMs, and flash memories. Examples of program instructions include not only machine language codes such as those generated by a compiler, but also high-level language codes that can be executed by a computer using an interpreter or the like. The hardware devices described above may be configured to operate as one or more software modules to perform the operations of the present disclosure, and vice versa.

Furthermore, the aforementioned focus control method and focus size control method may also be implemented in the form of a computer program or application stored on a recording medium and executed by a computer.

The above description of the present application is for illustrative purposes only, and those skilled in the art will understand that the present application can be easily modified into other specific forms without altering the technical spirit or essential characteristics of the present application. Therefore, the embodiments described above should be understood as illustrative in all respects and not restrictive. For example, each component described as a single component may be implemented in a distributed manner, and similarly, components described as distributed may be implemented in a combined manner.

The scope of the present application is defined by the claims described below rather than the detailed description above, and all changes or modifications derived from the meaning and scope of the claims and their equivalents should be construed as being included within the scope of the present application.

1. An ultrasound output device capable of controlling a focus, comprising:
an ultrasound output unit including a plurality of output channels; and
a control unit for controlling a focus generated by an ultrasound signal by controlling a delay time of the ultrasound signal output through the plurality of output channels and whether to activate the plurality of output channels.

2. The ultrasound output device of claim 1, wherein the control unit includes a grouping unit that groups the plurality of output channels to generate a plurality of channel groups.

3. The ultrasound output device of claim 2, wherein the control unit includes a multi-focus control unit that controls the delay time of a plurality of ultrasound signals output from each of the plurality of channel groups to generate a plurality of foci.

4. The ultrasound output device of claim 2, wherein the control unit includes a focus size control unit that controls the delay time of the plurality of ultrasound signals output from each of the plurality of channel groups to generate at least one focus and controls the focus size of the at least one focus by controlling whether to activate each of the plurality of channel groups.

5. The ultrasound output device of claim 4, wherein the focus size control unit includes
a prediction unit that predicts a prediction focus size when at least one channel group is activated based on angle information for the at least one channel group among the plurality of channel groups, and
a determination unit that determines whether to activate each of the plurality of channel groups based on the prediction focus size.

6. The ultrasound output device of claim 5, wherein when one channel group of the plurality of channel groups is activated, the angle information is a focus angle for the one channel group, and
when at least two channel groups of the plurality of channel groups are activated, the angle information is a sum of at least two focus angles for each of the at least two channel groups.

7. The ultrasound output device of claim 5, wherein the determination unit sets a plurality of focus size levels based on the angle information, and determines whether to activate each of the plurality of channel groups based on one focus size level of the plurality of focus size levels corresponding to a target focus size.

8. The ultrasound output device of claim 5, wherein the focus size control unit further includes a pressure maintenance unit that maintains a pressure intensity of the ultrasound signal within a preset range depending on whether to activate each of the plurality of channel groups.

9. An ultrasound focus control method, comprising controlling a focus generated by an ultrasound signal by controlling a delay time of the ultrasound signal output through a plurality of output channels and whether to activate the plurality of output channels.

10. The ultrasound focus control method of claim 9, wherein the controlling of the focus includes
grouping the plurality of output channels to generate a plurality of channel groups,
controlling the delay time of the ultrasound signal output from each of the plurality of channel groups to generate a plurality of foci, and
controlling the delay time of the ultrasound signal output from each of the plurality of channel groups to generate at least one focus, and controlling the focus size of the at least one focus by controlling whether to activate each of the plurality of channel groups.

11. The ultrasound focus control method of claim 10, wherein the controlling of the focus size includes
predicting a prediction focus size when at least one channel group is activated based on angle information for the at least one channel group among the plurality of channel groups,
determining whether to activate each of the plurality of channel groups based on the prediction focus size, and
maintaining a pressure intensity of the ultrasound signal within a preset range depending on whether to activate each of the plurality of channel groups.
